(19) United States
(12) Patent Application Publication
(10) Pub. No.: US 2022/0381756 A1
(43) Pub. Date: Dec. 1, 2022

(54) COMPOST MONITORING DEVICE AND SYSTEM

(71) Applicant: EarthOffset Holdings Pty Ltd, St. Lucia (AU)

(72) Inventor: Ashley Jane Baxter, St Lucia (AU)

(21) Appl. No.: 17/772,233

(22) PCT Filed: Oct. 30, 2020
(86) PCT No.: PCT/AU2020/051181

§ 371 Date: Apr. 27, 2022

(30) Foreign Application Priority Data
Oct. 30, 2019 (AU) 2019904088

(51) Int. Cl. G01N 33/00; H04Q 9/00

(52) U.S. Cl. CPC G01N 33/0031; H04Q 9/00; H04Q 2209/43

(57) ABSTRACT

A compost monitoring device for use in monitoring properties of a body of compost, the compost monitoring device being configured to be positioned proximate to the body of compost in use, and including: a sensor array including sensors for sensing properties of gas proximate to the body of compost, the sensors including a temperature sensor for sensing a temperature of the gas, a humidity sensor for sensing a relative humidity of the gas, and a gas sensor for detecting a level of a gas compound emitted from the compost within the gas; a wireless transceiver for wirelessly communicating with another device located remotely from the body of compost; and a controller configured to obtain sensor data from the sensors; generate monitoring data using at least some of the sensor data, and cause the monitoring data to be transmitted to the other device using the wireless transceiver.

### BACKGROUND OF THE INVENTION

The present invention relates to a compost monitoring device for use in monitoring properties of a body of compost, and a compost monitoring system using said compost monitoring device.

### DESCRIPTION OF THE PRIOR ART

Composting can have a significant positive environmental impact but can be confusing, time-consuming, slow, and if done wrong, can produce odours and attract pests. It is desirable to provide users with a capability to conveniently monitor a composting process and to enable users to determine whether interventions may be required before problems can develop. Unfortunately, prior attempts at providing such a capability have been unsuitable for adoption by the majority of users.

Traditional compost monitoring systems have taken a variety of forms, ranging from commercial systems, small-scale composting systems, to hobby and DW solutions.

As far as commercial systems are concerned, these systems are typically manufactured and distributed by composting, waste management and/or agricultural equipment companies. These systems are usually configured for use with windrow composting operations and rely on the use of sophisticated sensor probes that are inserted into the compost, often only as needed to prevent corrosion of the probes. They are intended for use in large-scale commercial composting and are therefore robust but expensive.

On the other hand, small-scale composting systems have been provided in the form of tools or products intended for residential, community or otherwise small-scale composters. The extent of sophistication for this product category is often limited to thermometers and moisture meters that must be manually inspected. The price point is very low, however the quality is also relatively low and inconsistent.

Finally, there is a small existing community of users that construct their own compost monitoring systems for personal use, as a hobby or do-it-yourself (DIY) project. These systems tend to use basic off-the-shelf components and provide limited capability and longevity.

In general, currently available compost monitoring devices are either expensive, scientific-standard probes or simple garden thermometers. The main limitations of the former is that while they may take highly precise readings at the point of probe, these are not representative of the entire pile, cannot collect data continuously and are very delicate instruments. The limitations of the latter are their lack of sophistication, offering nothing but a gauge temperature reading.

The majority of commercial compost monitoring systems have relied on the use of probes that are inserted into the compost, usually for measuring temperature, although additional properties may be measured. Depending on the particular implementation, the probes may include integral displays or may transmit measured data to other devices. Although probes allow direct measurements of internal properties of the compost, the probe measurements only represent the properties in a single position and may not be representative of the overall properties throughout a mass of compost.

For example, if a probe is inserted into a region containing organic matter that is rapidly decomposing, this may result in a “hot-spot” having temperature and other properties that may be drastically different to that of other regions of the compost. Accordingly, traditional compost monitoring systems may require the use of a plurality of probes to allow averaging of the probe measurements and thereby reduce the sensitivity to spurious measurements due to hot-spots or the like. However, this tends to drive the complexity and cost of the system even higher.

The reference in this specification to any prior publication (or information derived from it), or to any matter which is known, is not, and should not be taken as an acknowledgment or admission or any form of suggestion that the prior publication (or information derived from it) or known matter forms part of the common general knowledge in the field of endeavour to which this specification relates.

### SUMMARY OF THE PRESENT INVENTION

In one broad form an aspect of the present invention seeks to provide a compost monitoring device for use in monitoring properties of a body of compost, the compost monitoring device being configured to be positioned proximate to the body of compost in use, the compost monitoring device including: a sensor array including sensors for sensing properties of gas proximate to the body of compost, the sensors including a temperature sensor for sensing a temperature of the gas, a humidity sensor for sensing a relative humidity of the gas, and a gas sensor for detecting a level of a gas compound emitted from the compost within the gas; a wireless transceiver for wirelessly communicating with another device located remotely from the body of compost; and a controller configured to obtain sensor data from the sensors, generate monitoring data using at least some of the sensor data, and cause the monitoring data to be transmitted to the other device using the wireless transceiver.

In one embodiment, the gas sensor is a methane gas sensor for detecting a level of methane within the gas.

In one embodiment, the gas sensor is configured for detecting respective levels of at least some of the following compounds within the gas: methane; hydrogen; ethanol; carbon monoxide; and butane.

In one embodiment, the gas sensor is a volatile organic compounds detector for detecting respective levels of a plurality of gas compounds emitted from the compost within the gas.

In one embodiment, the sensor array is positioned externally to a surface of the body of compost and exposed to the gas proximate to the body of compost in use.

In one embodiment, the compost monitoring device includes a support arrangement for supporting the compost monitoring device relative to a surface of the body of compost in use.

In one embodiment, the support arrangement is configured to engage with the surface of the body of compost in use.

In one embodiment, the support arrangement is configured to at least one of: rest on the surface of the body of compost in use; and be partially inserted into the surface of the body of compost in use.

In one embodiment, the support arrangement is configured to define a hollow volume extending between the sensor array and the body of compost in use.

In one embodiment, the support arrangement is configured so that gas compounds emitted from the surface of the body of compost are captured in the hollow volume and directed to the sensor array in use.

In one embodiment, the support arrangement includes a hollow stake configured to be partially inserted into the surface of the body of compost.

In one embodiment, the hollow stake includes: a proximal end that surrounds the sensor array; a distal end opposing the proximal end, the distal end being for insertion into the surface of the body of compost; and a sidewall extending between the proximal end and the distal end and defining the hollow volume.

In one embodiment, the sidewall is tapered from the proximal end to the distal end.

In one embodiment, the sidewall of the hollow stake is vented.

In one embodiment, the support arrangement includes a skirt extending from the sensor array and including a rim for engaging with the surface of the body of compost in use.

In one embodiment, the skirt is flared such that the hollow volume expands outwardly from the sensor array to the rim.

In one embodiment, the body of compost is provided in a container, and the support arrangement is configured to engage with a wall of the container to thereby support the compost monitoring device relative to the body of compost.

In one embodiment, the support arrangement includes a magnetic coupling arrangement for magnetically coupling the compost monitoring device to the wall.

In one embodiment, the support arrangement is removably attached to the compost monitoring device.

In one embodiment, the compost monitoring device includes a removable filter proximate to the sensor array.

In one embodiment, the compost monitoring device includes a removable power supply unit including a rechargeable battery and a power supply interface for supplying power to the compost monitoring device.

In one embodiment, the power supply interface includes at least one of: electrical contacts; and an inductive coupling interface.

In one embodiment, the compost monitoring device is configured to be powered by a separate solar power supply.

In one embodiment, the wireless transceiver is configured for wirelessly communicating with the other device using a Bluetooth wireless communication protocol.

In another broad form an aspect of the present invention seeks to provide a compost monitoring system for monitoring properties of a body of compost, the system including: a compost monitoring device as described above, the compost monitoring device being positioned proximate to the body of compost in use; and a gateway device located remotely from the body of compost but within wireless communications range of the compost monitoring device in use, the gateway device including: a gateway wireless transceiver for wirelessly communicating with the compost monitoring device; a gateway network interface for communicating with a processing system via a communications network; and a gateway controller configured to receive monitoring data from the compost monitoring device, and transmit at least some of the monitoring data to the processing system via the communications network.

In one embodiment, the system further includes a server processing system, the server processing system being configured to: receive monitoring data from the gateway device; and determine a condition of the body of the compost based on received monitoring data.

In one embodiment, the server processing system is configured to allow a user processing system to access monitoring data from the server processing system via the Internet.

In one embodiment, the server processing system is configured to allow the user processing system to access the monitoring data using one of a web portal and an application programming interface.

In one embodiment, the server processing system is configured to generate one or more condition indicators indicative of the condition of the body of the compost and allow the user processing system to access the condition indicators.

In one embodiment, the server processing system is configured to: determine whether any user interventions are required based on the condition of the body of the compost; in the event of a determination that one or more user interventions are required, generate a user intervention indication indicative of the one or more user interventions; and transmit the user intervention indication to the user processing system.

In one embodiment, the user interventions include at least one of: turning the body of compost; adding moisture to the body of compost; and adding organic matter to the body of compost.

In one embodiment, the server processing system is configured to obtain, from a meteorological data source, meteorological data for a geographical region corresponding to a location of the body of compost.

In one embodiment, the server processing system is configured to obtain the meteorological data based on location data for at least one of the compost monitoring device, the gateway device, and the remote user device.

In one embodiment, the meteorological data includes at least one of: a relative humidity observation; and a temperature observation.

In one embodiment, the server processing system is configured to determine the condition of the body of the compost based on received monitoring data and the meteorological data.

In one embodiment, the server processing system includes a plurality of processing devices in a cloud architecture.

In one embodiment, the gateway network interface includes a wireless network interface for wirelessly communicating with the server processing system using a wireless communications network.

In one embodiment, the gateway network interface includes a wireless modem for wirelessly communicating with the server processing system via the Internet.

In one embodiment, the gateway network interface includes a wireless access point for allowing a user device to wirelessly connect to the gateway device to allow the local user device to access monitoring data directly from the gateway device.

In one embodiment, the compost monitoring device includes a removable power supply unit including a rechargeable battery and the gateway device further includes a battery charging interface for recharging the battery of the removable power supply unit.

It will be appreciated that the broad forms of the invention and their respective features can be used in conjunction, interchangeably and/or independently, and reference to separate broad forms is not intended to be limiting.

### BRIEF DESCRIPTION OF THE DRAWINGS

Various examples and embodiments of the present invention will now be described with reference to the accompanying drawings, in which:

FIG. 1A is a schematic diagram of a first example of a compost monitoring device for use in monitoring properties of a body of compost, in use;

FIG. 1B is a schematic diagram of a second example of a compost monitoring device, in use;

FIG. 2 is a schematic diagram of functional elements of an example of a compost monitoring device;

FIG. 3A is a first perspective view of an example of a compost monitoring device;

FIG. 3B is a second perspective view of the compost monitoring device of FIG. 3A;

FIG. 3C is an exploded view of the compost monitoring device of FIG. 3A;

FIG. 3D is a cross section view of the compost monitoring device of FIG. 3A; and

FIG. 4 is a schematic diagram of functional elements of an example of a gateway device for use in a system for monitoring properties of a body of compost;

FIG. 5 is a schematic diagram of an example of a distributed processing system architecture for use in a system for monitoring properties of a body of compost;

FIG. 6 is a schematic diagram of an example of a server processing system; and

FIG. 7 is a schematic diagram of an example of a user processing system.

### DETAILED DESCRIPTION OF THE PREFERRED EMBODIMENTS

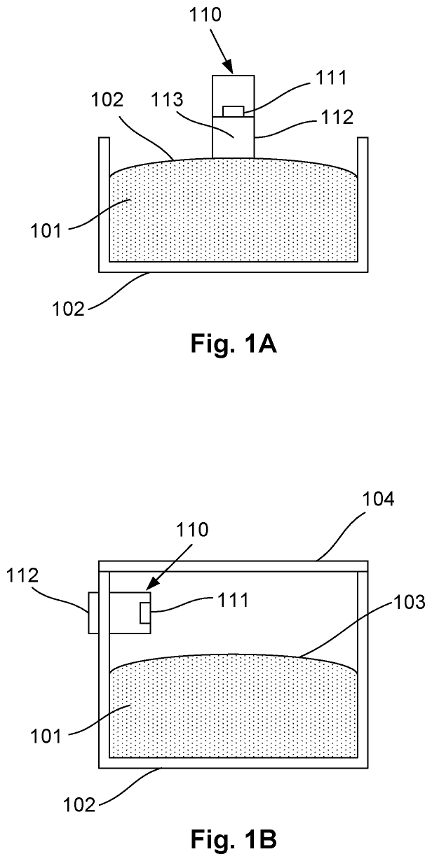

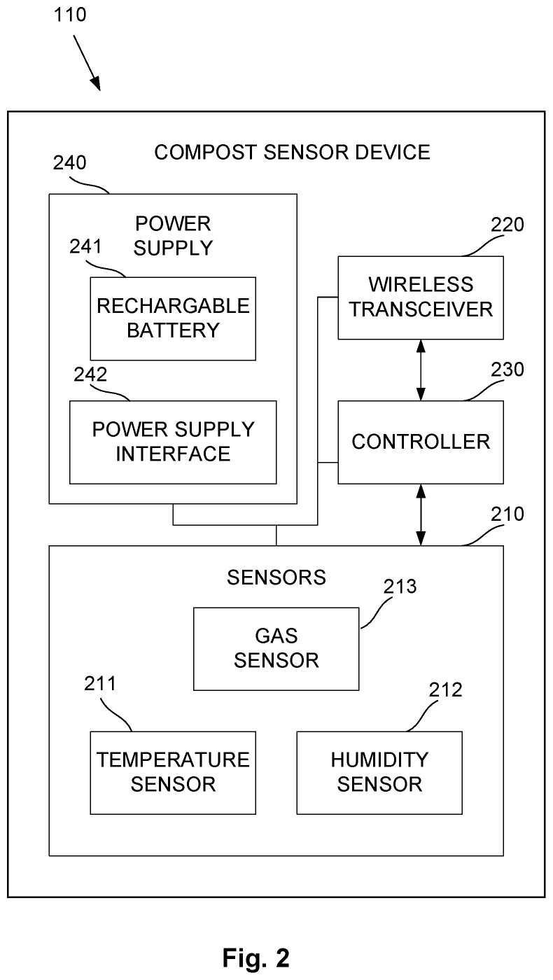

An example of a compost monitoring device 110 for use in monitoring properties of a body of compost 101 will now be described with reference to FIG. 1A, which shows a first example of the compost monitoring device 110 in use, and FIG. 2, which shows a schematic representation of functional elements of the compost monitoring device 110.

It should be appreciated that, throughout this description, the term “compost” is used to generally refer to organic matter throughout the various stages of a composting process, including fresh organic matter that has not yet started to decompose, partially decomposed organic matter, and fully decomposed compost products.

For the purpose of this example, the body of compost 101 is assumed to be provided in a suitable container 102 such as a compost bin or the like, although this is not essential. For example, the body compost 101 may be provided as a simple heap on the ground or any other surface. It is also assumed that the body of compost 101 has an accessible surface 102. In this example, the body of compost 101 is uncovered, but once again this is not essential. For instance, the alternative example shown in FIG. 1B, to be described further in due course, shows a covered body of compost 101.

In any event, the compost monitoring device 110 is configured to be positioned proximate to the body of compost 101 in use, and includes a sensor array 111 including a plurality of sensors for sensing properties of gas proximate to the body of compost 101.

With regard to FIG. 2, the sensors 210 include a temperature sensor 211 for sensing a temperature of the gas, a humidity sensor 212 for sensing a relative humidity of the gas, and a gas sensor 213 for detecting a level of a gas compound emitted from the compost within the gas.

The compost monitoring device 110 further includes a wireless transceiver 220 for wirelessly communicating with another device located remotely from the body of compost 101, and a controller 230. The controller 230 is particularly configured to obtain sensor data from the sensors 111, generate monitoring data using at least some of the sensor data, and cause the monitoring data to be transmitted to the other device using the wireless transceiver 220.

It will be appreciated that the use of gas based sensors 210 in the compost monitoring device 110 may provide a number of advantages compared to current probe based compost monitoring solutions. The compost monitoring device 110 effectively collects monitoring data from gas emitted from the surface 103 of the body of compost 101, making it easier to use and lower cost. It continuously and wirelessly transmits the collected temperature, humidity and gas compound level data to another device where it can be analysed to allow useful monitoring information to be provided to a user, including the option of recommending user interventions based on the monitoring data.

The use of gas based sensors 210 enables monitoring of sensor data that is more representative of the average properties of the body of compost 101, since the measurements of temperature, humidity and gas compound levels will tend to relate to an average of the properties within the gas proximate to the body of compost 101, as opposed to the single-point measurements obtained by probe sensors which can be subject to spurious “hot-spot” measurements.

Furthermore, the use of gas based sensors 210 avoids the need for the sensors to be in contact with the compost, which can substantially decrease the risk of corrosion of the sensors. Traditional probe sensors may easily corrode when left inserted in the compost, whereas the sensors 210 will typically be separated from the surface 103 of the body of compost 101 and so not exposed to the same risk of corrosion.

It will be appreciated that this can allow the compost monitoring device 110 to be used for continuous monitoring, in contrast to many conventional probe based solutions which rely on users to manually insert probes when measurements are required.

The gas based sensors 210 may be selected from commercially available and low cost sensors, thereby allowing a far more affordable price point to be achieved.

It is acknowledged that the use of gas based sensors may sacrifice some measurement accuracy compared to direct contact probe sensors. However, this accuracy can be at least partially recovered through the use of continuous measurement, and it will also be understood that the above discussed averaging effect will tend to result in more reliable representative measurements in any event.

The compost monitoring device 110 may be used to transmit monitoring data to a range of different types of other devices, depending on the implementation. In some cases, the compost monitoring device 110 may be configured to transmit monitoring data directly to a user device, such as a mobile phone, tablet, laptop computer or the like. However, as will be described in further detail below, it may be desirable to configure the compost monitoring device 110 to operate using a limited on-board power supply, such as a rechargeable battery, and in order to conserve power, the wireless transceiver 220 may be implemented using a low-power wireless transmission protocol, which may have a restricted wireless transmission range. This may result in monitoring data only being transmitted when the user device is within wireless transmission range of the compost monitoring device 110.

If continuous monitoring of the compost is desired, it may be preferable to use the compost monitoring device 110 together with a separate gateway device located remotely from the body of compost 101 but within wireless communications range of the compost monitoring device 110 in use. Since the gateway device may be located remotely from the body of compost 101, it may be more readily connected to a constant power source such as mains electricity. This may support the use of a higher-power wireless network interface capable of communicating with a local area network or the Internet, to thereby allow the monitoring data to be continuously transmitted to other devices.

The compost monitoring device 110 may be used in a compost monitoring system, which may include the gateway device together with a server processing system for receiving and processing the monitoring data and a user device for allowing a user to access the monitoring data and information derived therefrom by the server processing system. A detailed example of such a compost monitoring system will be described in due course.

In the meantime, a range of optional features of preferred embodiments of the compost monitoring device 110 itself will now be described.

As far as the sensors 210 are concerned, as mentioned above, these may be implemented using commercially available and low-cost gas based sensors. The temperature sensor 211 and the humidity sensor 212 may be provided as separate sensors or using an integrated temperature and humidity sensor.

In any event, the measurement of temperature of gas emitted from the surface 103 of the body of compost 101 can be indicative of the internal temperature of the body of compost 101, which in turn can reflect the stage and quality of the composting process. Similarly, the measurement of humidity, typically as a relative humidity percentage, can be indicative of the internal moisture level of the body of compost 101. High relative humidity levels fluctuating in the range of 90-100% are commonly encountered throughout the decomposition process, whereas lower humidity levels may indicate that the compost 101 is too dry for effective decomposition.

As far as the gas sensor 213 is concerned, this may be configured to detect the level of a particular gas compound that is indicative of the composing process. The level of the gas compound will typically be detected as a relative concentration of the gas compound within the gas, although this is not essential. For instance, the gas sensor 213 may be configured to detect the presence of a gas beyond a predetermined threshold, as opposed to a concentration.

In some embodiments, the gas sensor 213 may be provided as a methane gas sensor for detecting a level of methane within the gas. It will be appreciated that the level of methane emitted from the body of compost will provide a useful indicator of the properties of the composting process. For example, low methane levels of less than 50 ppm are indicative of a good composting process, whereas higher methane levels may indicate the presence of a problem that may require user intervention. In other examples, methane levels may be monitored over time in order to derive insights from relative increases and decreases rather than individual readings.

Although measurements of temperature, humidity and methane levels may be sufficient to allow effective monitoring of a composting process and to facilitate users to make timely interventions as required, it may nevertheless be desirable to obtain additional measurements using the compost monitoring device.

For instance, more comprehensive measurements of the levels of gas compounds emitted from the compost 101 may be achieved through the use of a more sophisticated gas sensor 213. For example, the gas sensor 213 may be configured for detecting respective levels of a range of different compounds within the gas, such as methane, hydrogen, ethanol, carbon monoxide, butane, or combinations thereof. Each of these compounds may be emitted from the compost as by-products of the composting process, and the measurement of levels of more than one of these compounds can provide further information regarding the quality of the ongoing decomposition of the organic material in the compost 101. It will be appreciated that these different compounds may be measured in a range of different combinations and sensitives depending on the implementation.

In one specific example, the gas sensor 113 may be provided in the form of a volatile organic compounds (VOC) detector for detecting respective levels of a plurality of gas compounds emitted from the compost within the gas. Such a VOC sensor may be capable of detecting hydrogen, ethanol, carbon monoxide, butane and methane in varying sensitivities.

As mentioned above, the sensors 210 are provided in a sensor array 111 on the compost monitoring device. Preferably, the sensor array 111 will be positioned externally to the surface 103 of the body of compost 101 and exposed to the gas proximate to the body of compost 101 in use. It is desirable to ensure that the sensor array 111 is separated from the surface 103 of the body of compost 101 so that the sensors 210 are located in a suitable position for measuring properties of the gas proximate to the body of compost 101 as opposed to properties of the surface 103 itself, in order to achieve the above discussed advantages associated with gas based sensors compared to contact or probe based sensors.

Accordingly, in preferred embodiments, the compost monitoring device 110 may include a support arrangement 112 for supporting the compost monitoring device 110 relative to the surface 101 of the body of compost 103 in use. In general, the support arrangement 112 will be used to ensure that the sensor array 111 is positioned with an appropriate separation from the surface 103 of the body of compost 101 to allow effective gas sensor measurements.

Suitable support arrangements 112 may take a variety of forms, but in general, these may be separated into two types, as illustrated in the examples of FIGS. 1A and 1B.

FIG. 1A shows a first type of support arrangement 112, which is configured to engage with the surface 103 of the body of compost 101 in use. The support arrangement 112 may be configured to rest on the surface 103 or be partially inserted into the surface 103 in use. Either way, the support arrangements 112 may be used to position the sensor array 111 separately from the surface 103. It will be appreciated that the distance of separation between the sensor array 111 and the surface 103 may be controlled by configuring the support arrangement accordingly.

The particular structural configuration of this first type of support arrangement 112 can be varied depending on requirements. In some examples, the support arrangement 112 can be provided in the form of a simple stake or other elongate member that can be driven into the surface 103 of the compost 101. In other examples, the support arrangement 112 may include a plurality of elongate members. For instance, three elongate members may be provided as legs of a tripod that can be rested on or partially inserted into the surface 103.

As shown in the example of FIG. 1A, the support arrangement 112 may be configured to define a hollow volume 113 extending between the sensor array 111 and the body of compost 101 in use. This may be achieved by forming the support arrangement in an open ended prismatic shape, such as a cylinder.

Preferably, such a support arrangement 112 may be configured so that gas compounds emitted from the surface 103 of the body of compost 101 are captured in the hollow volume and directed to the sensor array 111 in use. It will be appreciated that this can help to ensure that the sensor array 111 will be exposed to the gas compounds emitted from the surface 103. This can be especially useful when the compost monitoring device 110 is used to monitor properties of a body of compost 101 that is uncovered as shown in FIG. 1A, where the emitted gas may be exposed to environmental effects, such as strong winds which could blow gas compounds away from the surface before they are able to reach the sensor array 111.

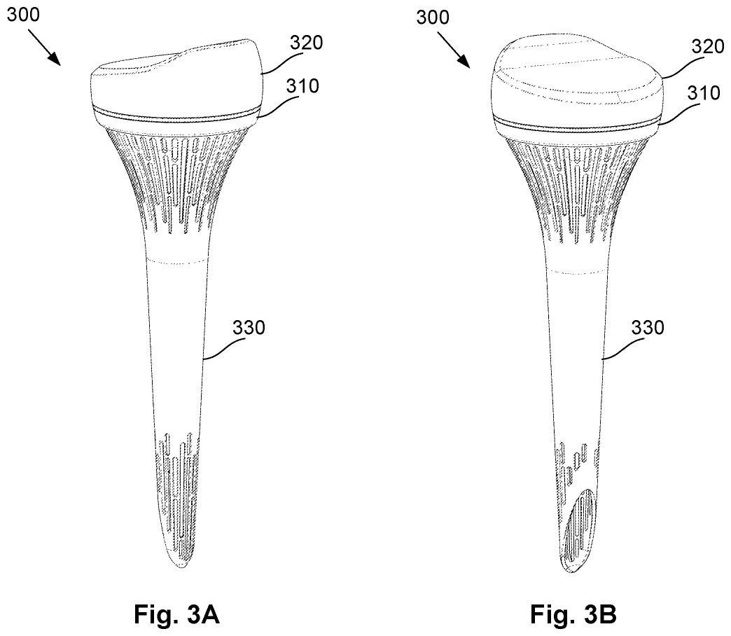

A specific example of a compost monitoring device 300 having a support arrangement 112 of this type is shown in FIGS. 3A to 3D. In this example, the compost monitoring device 300 includes a main sensor unit 310 including the sensor array 111, which is coupled to a power supply unit 320 and a hollow stake 330 that functions as the support arrangement 112.

In this case, the hollow stake 330 is configured to be partially inserted into the surface 103 of the body of compost 101, and defines a hollow volume for directing gas from the surface to the sensor unit 310. As best seen in FIGS. 3C and 3D, the hollow stake 330 includes a proximal end 331 that surrounds the sensor array 111, a distal end 332 opposing the proximal end 331 and a sidewall 333 extending between the proximal end 331 and the distal end 332 and defining the hollow volume.

The distal end 332 is configured for insertion into the surface 103 of the body of compost 101, and may include a sharp tip in the fashion of a hypodermic needle to aid insertion. The sidewall 333 may also be tapered from the proximal end 331 to the distal end 332, which can also aid insertion. In this case, the sidewall 333 also includes a funnel portion near the proximal end 331 in which the cross section reduces between the sensor unit 310 and a section partially along the length of the sidewall 333 where the sidewall 333 adopts a constant taper towards the distal end 332.

As shown in this example, the sidewall of the hollow stake 330 may also be vented. In this case, a first pattern of vents 334 is provided around the proximal end 331, which allows gas directed to the sensor array 111 to escape from the hollow volume, to be replaced as more gas is emitted from the compost 301 and flows through the hollow volume. A second pattern of vents 335 is provided around the distal end 332, which allows some gas to also be captured from compost surrounding the inserted portion of the hollow stake 330 in use. It should be appreciated that vents 334, 335 are not essential, but they may be used to modify the flow of gas into and out of the hollow volume, which may be advantageous depending on the compost monitoring device design.

It will be appreciated that other examples of support arrangements 112 may generally include a skirt extending from the sensor array 111, with the skirt including a rim for engaging with the surface 103 of the body of compost 101 in use. This corresponds to the schematic arrangement shown in FIG. 1A. The rim may rest upon the surface 103 or may be partially embedded into the surface 103 to more securely engage with the surface 103. In some examples, the skirt may be flared such that the hollow volume 113 expands outwardly from the sensor array 111 to the rim. Such a flared skirt may be used to capture gas from an enlarged area of the surface 103 of the compost 101, compared to the size of the sensor array 111 and other components of the compost monitoring device.

However, as mentioned above, the example of FIG. 1B illustrates a second type of support arrangement 112 that may be used. In particular, when the body of compost 101 is provided in a container 102, the support arrangement 112 may be configured to engage with a wall of the container 102 to thereby support the compost monitoring device 110 relative to the body of compost 101. Accordingly, this second type of support arrangement 112 utilises existing structure of the container 102, which removes the need to provide a robust support structure.

However, one downside of this type of support arrangement 112 compared to the first type of support arrangement 112 described with regard to FIG. 1A is that this removes some control over the separation distance between the sensor array 111 and the surface 103 of the compost 103. Furthermore, another downside is that this second type of support arrangement 112 does not provide a hollow volume for directing gas to the sensor array 111 and therefore the sensor array 111 may not necessarily be exposed to gas emitted from the compost 103 if it is blown away by wind, for example. Nevertheless, the impact of these downsides may be reduced when this second type of support arrangement 112 is used with an enclosed compost container, such as a compost bin including a lid 104 or the like. Such an enclosed environment will ensure that gas emitted from the compost 101 will not be lost due to wind before the sensor array 111 is exposed, and will help to reduce any sensitivity to separation distance between the sensor array 111 and the surface 103.

Examples of support arrangements 112 configured to engage with a wall of the container 102 may include simple fastening arrangements, such as the inclusion of mounting points for allowing the compost monitoring device 110 to be fastened to the wall of the container 102 using threaded screws or the like. However, this may require permanent alterations to the container 102, and may also make removal of the compost monitoring device 110 difficult.

A more flexible implementation of a support arrangement 112 of the type shown in FIG. 1B may include the use of a magnetic coupling arrangement for magnetically coupling the compost monitoring device 110 to the wall of the container 102. For example, the support arrangement 112 may be provided with a permanent magnet that can be positioned outside the container 102, whilst the compost monitoring device 110 may include a ferromagnetic material such that it will be magnetically attracted to the support arrangement 112, so that the wall of the container 102 is sandwiched between the support arrangement 112 and the compost monitoring device 110 to effectively secure the compost monitoring device 110 to the container 102. It will be appreciated that this will allow the compost monitoring device 110 to be selectively removed or repositioned as required, without permanent alterations to the container 102.

In any event, embodiments of the support arrangement 112, of either of the types described above, may be configured to be removably attached to the compost monitoring device 110. For example, the hollow stake 330 which functions as the support arrangement 112 in the compost monitoring device 300 of FIGS. 3A to 3D can be attached by a bayonet connection or any other suitable removable attachment mechanism.

As mentioned above, the compost monitoring device 300 of FIGS. 3A to 3D may also include an optional filter 340 proximate to the sensor array 111, as shown in FIGS. 3C and 3D. This filter 340 may be provided to cover the sensor array 111, for example. In this case, the filter 340 extends across the vents 334 near the proximal end 331 of the hollow stake 330. This may be used to prevent or reduce the ingress of particles of material or other undesirable objects, insects or the like, into the hollow stake near the sensor array 111. Some embodiments of the filter 340 may be designed for replacement and may have an open cell construction using a compostable biopolymer filter to allow environmentally friendly disposal of replaced filters 340. Other embodiments of the filter 340 may be washable and serviceable.

Turning back to FIG. 2, which shows functional elements of the compost sensor device 110, it will be noted that embodiments of the compost sensor device 110 may further include a power supply 240 for supplying electrical power to the sensors 210, wireless transceiver 220 and the controller 230. Alternative embodiments may be powered by an external power source, such as via a wired connection to mains electricity or a separate solar power supply. However, it may be desirable to provide an on-board power supply without requiring wired connections to other power sources.

In the embodiment of FIG. 2, the power supply 240 includes a rechargeable battery 241 and a power supply interface 242, which may be used to connect the power supply 240 to other elements of the compost monitoring device 300, and/or to facilitate recharging of the battery 241. In the example compost monitoring device 300 of FIGS. 3A to 3D, the compost monitoring device 300 specifically includes a power supply unit 320 that is removable, as can be seen in the exploded view of FIG. 3C.

As shown in the section view of FIG. 3D, the removable power supply unit 320 includes rechargeable batteries 241 and a power supply interface 242 in the form of electrical contacts. In this case, when the power supply unit 320 is attached to the sensor unit 310 of the compost monitoring device, the electrical contacts of the power supply interface 242 are brought into contact with corresponding electrical contacts of the sensor unit 310 to power the functional elements of the compost monitoring device 110 provided in the sensor unit 310, including the sensors, 210, the wireless transceiver 220 and the controller 230.

The power supply unit 320 can be recharged by removing it from the sensor unit 310 and attaching it to a suitably configured recharging unit (not shown) having corresponding electrical contacts connected to an external power source such as mains electricity. As will be discussed in further detail in due course, recharging may also be facilitated using a gateway device in some embodiments of the monitoring system.

Although a power supply interface 242 having physical electrical contacts has been described, it should be appreciated that other types of interfaces may be provided. For example, embodiments may have a power supply interface 242 configured for wireless inductive charging or other contactless energy transfer mechanisms. This can remove the need for electrical contacts penetrating through an enclosure of the device.

It will be appreciated that there are a number of environmental considerations that should be accounted for in the design of the compost monitoring device 300, particularly with regard to its use in proximity to the body of compost 101. For instance, the temperature and humidity of small-scale composting operations can vary significantly but would usually be expected to be substantially higher than ambient conditions, so the physical design of the device and its hardware elements should be selected accordingly.

In addition, organic compounds may be continuously emitted by the compost, including methane, ethanol and propane. Methane is the most abundant, reaching high levels of up to 500 ppm. Similarly high levels of sulphuric compounds, which may combine with water vapour, may also be present. Furthermore, the pH of the compost may range from 5-9 throughout the compositing process. Accordingly, these conditions should also be factored into the selection of materials used in the manufacture of external components of the device, and in the sealing of the components.

For example, the hollow stake 330 will preferably be formed from a material that is suitable for withstanding prolonged direct contact with the compost without corroding, reacting or breaking down due to exposure to the harsh compost environment. Plastic materials commonly used in the manufacture of compost bins, such as UV stabilised high density polyethylene (HDPE) or the like may be especially suitable in this regard, although it will be appreciated that a range of other suitable materials may be used, such as stainless steel. Similar material selection considerations may be applied to the design of external housings of other components, such as the sensor unit 320 and the power supply unit 330. It should be noted that these components will not be in direct contact with the compost 101, but may nevertheless be exposed to increased temperature, humidity and volatile compounds in proximity to the compost 101.

With regard to the wireless transceiver 220, in preferred embodiments this will be configured for wirelessly communicating with other devices using a low-power wireless communication protocol, such as Bluetooth. It will be appreciated that the wireless communication protocol and its data transmission range and rate may have a significant impact on the power drain rate of the compost monitoring device 110 and therefore should be selected with regard to the available power supply capacity.

The controller 230 can be of any appropriate form, but in one example includes at least one microprocessor, a memory, and an external interface, which may be interconnected by a bus. In this case, the external interface is connected to the sensors 110 and the wireless transceiver 220. In use, the microprocessor may execute software instructions stored in the memory to allow the required processes to be performed. Accordingly, it will be appreciated that the controller 230 may be formed from any suitable processing system arrangement and could include any electronic processing device such as a microcontroller unit, microchip processor, logic gate configuration, firmware optionally associated with implementing logic such as an FPGA (Field Programmable Gate Array), or any other electronic device, system or arrangement.

As mentioned above, whilst the compost monitoring device 110 may be capable of directly communicating with a user device such as a mobile phone, this may only allow monitoring data to be transmitted to the user device when it is within wireless transmission range and thereby prevent continuous monitoring of properties of the compost.

Accordingly, it may be preferable to provide the compost monitoring device 110 as part of a compost monitoring system for monitoring properties of the body of compost 101. In one example, such a system will include the compost monitoring device 110 that is positioned proximate to the body of compost 101 in use as described above, together with a gateway device that is located remotely from the body of compost 101 but within wireless communications range of the compost monitoring device 110 in use.

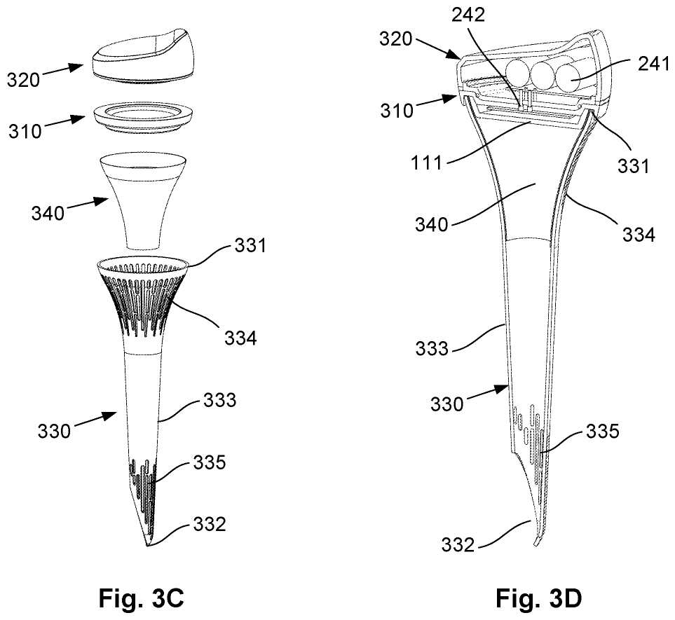

FIG. 4 shows a schematic representation of functional elements of an example of a suitable gateway device 410. With regard to FIG. 4, embodiments of the gateway device 410 will generally include a gateway wireless transceiver 420 for wirelessly communicating with the compost monitoring device 110 via its wireless transceiver 220, a gateway network interface 430 for communicating with a processing system via a communications network, and a gateway controller 440 configured to receive monitoring data from the compost monitoring device 110 and transmit at least some of the monitoring data to the processing system via the communications network.

In preferred embodiments, the gateway network interface 430 will typically include a wireless local area network (LAN) interface 431 for wirelessly communicating with the server processing system using a wireless communications network. However, the gateway network interface 430 may additionally or alternatively include a wireless modem 432 for wirelessly communicating with the server processing system via the Internet, and/or a wireless access point for allowing a user device to wirelessly connect to the gateway device to allow the local user device to access monitoring data directly from the gateway device, without requiring communications using a separate wireless LAN.

The gateway device 410 will also include a power supply 450, typically drawing from an external power source such as mains electricity. It will be appreciated that the gateway device 410 does not need to be provided proximate to the body of compost 101 as per the compost monitoring device 110, and the gateway device 410 can be readily positioned near an external power source whilst being within wireless transmission range of the compost monitoring device 110.

As discussed above, examples of the compost monitoring device 300 may include a removable power supply unit 320 including rechargeable batteries 241 and a power supply interface 242, which can be used for recharging the batteries 241. In some examples, the gateway device 410 may further include a charging interface 460 for use in recharging the batteries 241 of the removable power supply unit 320. It will be appreciated that the charging interface 460 may include electrical contacts corresponding to those provided in the removable power supply unit 320 to facilitate this. Whilst not essential, this configuration of the gateway device 410 can provide a convenient solution for recharging the power supply unit 320 without the need to provide additional recharging equipment.

It should also be appreciated that embodiments of the system may include a plurality of compost monitoring devices 110 which may be in wireless communication with one or more gateway devices 410. The use of multiple compost monitoring devices 110 can allow for more accurate monitoring of a large body of compost 101 by averaging the monitoring data obtained, or can allow for multiple bodies of compost 101 to be monitored collectively. The system may be expanded through the addition of compost monitoring devices 110 over time to suit the scale of the users composting operations.

Although the gateway device 410 may be configured to transmit the monitoring data to any form of processing system, such as a user processing system including a mobile phone or personal computer, typically the system further includes a server processing system that is configured to receive monitoring data from the gateway device and determine a condition of the body of the compost 101 based on received monitoring data. It will be appreciated that such an arrangement can allow the monitoring data to be centrally processed using analytical models of the composting process to determine the condition of the compost 101. In some examples, the server processing system may be implemented using a plurality of processing devices in a cloud architecture.

In preferred embodiments, the server processing system may be configured to allow a user processing system to access monitoring data from the server processing system via the Internet. In some examples, the server processing system may be configured to allow the user processing system to access the monitoring data using a web portal or an application programming interface.

In preferred embodiments, the server processing system will be configured to process the monitoring data to generate one or more condition indicators that are indicative of the condition of the body of the compost, and allow the user processing system to access the condition indicators. For example, the condition indicators may be in the form of parameters representing the progress or quality of the ongoing compositing progress, derived from the monitoring data.

Furthermore, the server processing system may be configured to determine whether any user interventions are required based on the condition of the body of the compost. Then, in the event of a determination that one or more user interventions are required, the server processing system may generate a user intervention indication indicative of the one or more user interventions and transmit the user intervention indication to the remote user device.

For example, the user interventions may include at least one of: turning the body of compost, adding moisture to the body of compost, and adding organic matter to the body of compost. In some examples, the user intervention indications may provide specific details such as a volume of water to be added, or a type of organic matter to be added, based on other known properties of the compost pile that may be provided by the user via the user processing system.

The server processing system may also utilise additional data beyond that obtained from the compost monitoring sensor 110 and the user. For instance, the server processing system may be configured to obtain, from a meteorological data source, meteorological data for a geographical region corresponding to a location of the body of compost. In one example, the server processing system may be configured to obtain the meteorological data based on location data for at least one of the compost monitoring device, the gateway device, and the remote user device. The meteorological data may include relative humidity observations and/or temperature observations. The server processing system may thus be configured to determine the condition of the body of the compost based on received monitoring data and the meteorological data.

It will be appreciated that the use of meteorological data in this regard may be used in the interpretation of the monitoring data when determining the condition of the compost. For instance, the measured temperature of the gas proximate to the compost may be compared to temperature observations to determine a temperature difference that may be used to estimate a relative temperature of the compost. Similarly, the measured relative humidity of the gas proximate to the compost may be compared to humidity observations to assist in estimating the moisture content of the compost.

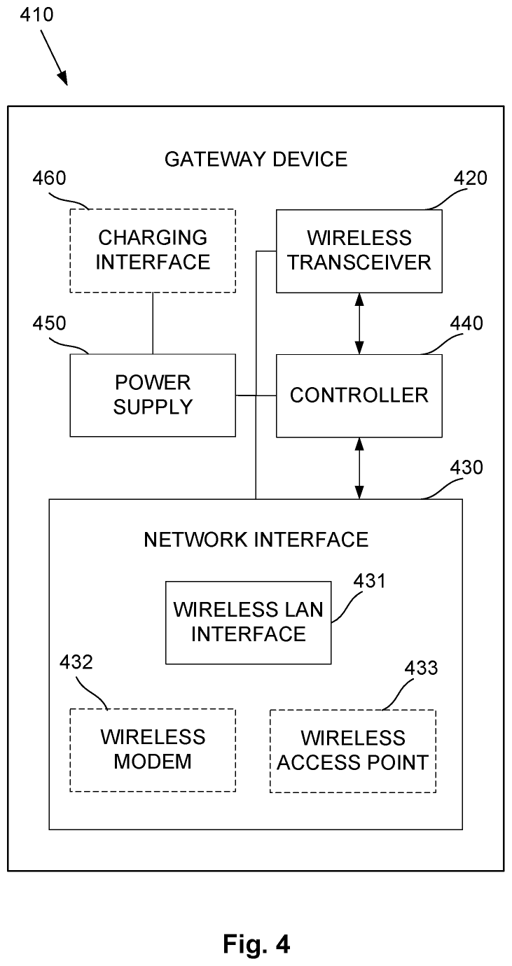

In any event, it will be appreciated that the compost monitoring device 110 may be used in a compost monitoring system to provide continuous monitoring and preferably analysis and feedback to the user. It will be appreciated that suitable examples of the system may be implemented by one or more processing systems operating as part of a distributed architecture, an example of which will now be described with reference to FIG. 5.

In this example, the arrangement includes a number of processing systems 501, 503 along with compost monitoring and gateway devices 110, 410, each interconnected via one or more communications networks, such as the Internet 502, and/or a number of local area networks (LANs) 504.

It will be appreciated that the configuration of the networks 502, 504 are for the purpose of example only, and in practice the processing systems 501, 503 and compost monitoring and gateway devices 110, 410 can communicate via any appropriate mechanism, such as via wired or wireless connections, including, but not limited to mobile networks, private networks, such as an 802.11 networks, the Internet, LANs, WANs, or the like, as well as via direct or point-to-point connections, such as Bluetooth, Zigbee or the like.

The nature of the processing systems 501, 503 and their functionality will vary depending on their particular requirements. In one particular example, the processing systems 501, 503 represent servers (such as for determining conditions indicative of the composting process based on the monitoring data) and clients (for allowing users to monitor the composting process), although this is not essential and is used primarily for the purpose of illustration. Whilst a number of clients, also referred to as user processing systems 503, are shown in FIG. 5, this is intended to illustrate the different potential configurations and network connections that may be utilised as opposed to indicating that multiple user processing systems 503 are necessary.

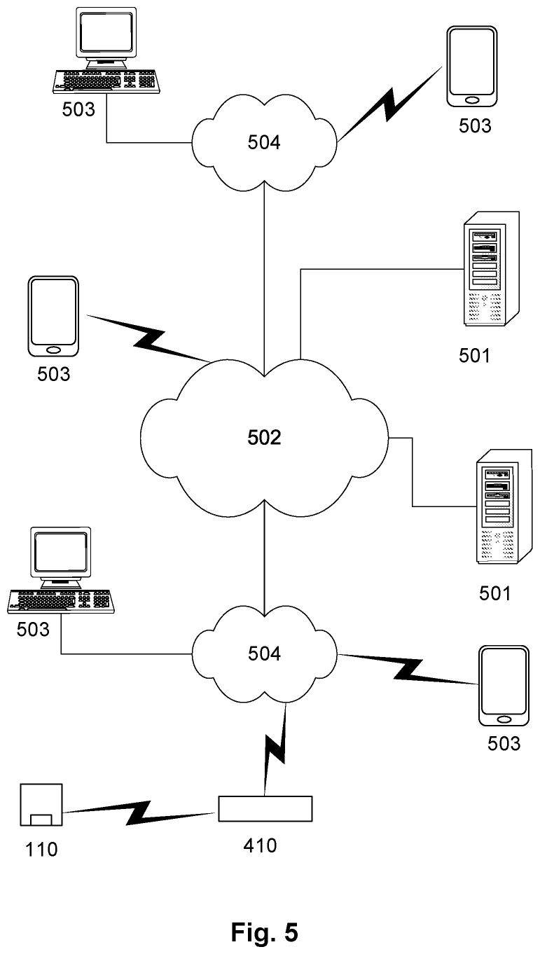

An example of a suitable server processing system 501 is shown in FIG. 6. In this example, the server processing system 501 includes an electronic processing device, such as at least one microprocessor 600, a memory 601, an optional input/output device 602, such as a keyboard and/or display, and an external interface 603, interconnected via a bus 604 as shown. In this example, the external interface 603 can be utilised for connecting the processing system 601 to peripheral devices, such as the communications networks 502, 504, databases 611, other storage devices, or the like. Although a single external interface 603 is shown, this is for the purpose of example only, and in practice multiple interfaces using various methods (e.g. Ethernet, serial, USB, wireless or the like) may be provided.

In use, the microprocessor 600 executes instructions in the form of applications software stored in the memory 601 to perform required processes, such as communicating with other processing systems 501, 503 or the compost monitoring and gateway devices 110, 410 depending on the system configuration. Thus, actions performed by a server processing system 501 are performed by the processor 600 in accordance with instructions stored as applications software in the memory 601 and/or input commands received via the I/O device 602, or commands received from other processing systems 501, 503. The applications software may include one or more software modules, and may be executed in a suitable execution environment, such as an operating system environment, or the like.

Accordingly, it will be appreciated that the server processing systems 501 may be formed from any suitable processing system, such as a suitably programmed computer system, PC, web server, network server, or the like. In one particular example, the server processing system 501 is a standard processing system such as a 32-bit or 64-bit Intel Architecture based processing system, which executes software applications stored on non-volatile (e.g., hard disk) storage, although this is not essential. However, it will also be understood that the server processing systems 501 could be or could include any electronic processing device such as a microprocessor, microchip processor, logic gate configuration, firmware optionally associated with implementing logic such as an FPGA (Field Programmable Gate Array), or any other electronic device, system or arrangement.

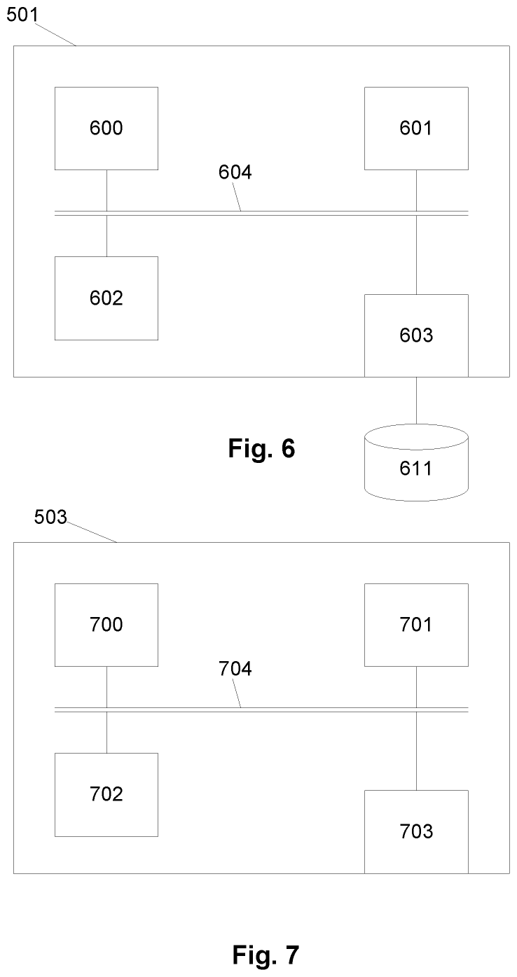

As shown in FIG. 7, in one example, the user processing systems 503 include an electronic processing device, such as at least one microprocessor 700, a memory 701, an input/output device 702, such as a keyboard and/or display, and an external interface 703, interconnected via a bus 704 as shown. In this example the external interface 703 can be utilised for connecting the user processing system 503 to peripheral devices, such as the communications networks 502, 504, databases, other storage devices, or the like. Although a single external interface 703 is shown, this is for the purpose of example only, and in practice multiple interfaces using various methods (e.g. Ethernet, serial, USB, wireless or the like) may be provided.

In use, the microprocessor 700 executes instructions in the form of applications software stored in the memory 701 to perform required processes, for example to allow communication with other processing systems 501, 503. Thus, actions performed by a user processing system 503 are performed by the processor 701 in accordance with instructions stored as applications software in the memory 702 and/or input commands received from a user via the I/O device 703. The applications software may include one or more software modules, and may be executed in a suitable execution environment, such as an operating system environment, or the like.

Accordingly, it will be appreciated that the user processing systems 503 may be formed from any suitable processing system, such as a suitably programmed PC, Internet terminal, laptop, hand-held PC, smart phone, PDA, tablet, or the like. Thus, in one example, the user processing system 503 is a standard processing system such as a 32-bit or 64-bit Intel Architecture based processing system, which executes software applications stored on non-volatile (e.g., hard disk) storage, although this is not essential. However, it will also be understood that the user processing systems 503 can be any electronic processing device such as a microprocessor, microchip processor, logic gate configuration, firmware optionally associated with implementing logic such as an FPGA (Field Programmable Gate Array), or any other electronic device, system or arrangement.

It will also be noted that whilst the processing systems 501, 503 are shown as single entities, it will be appreciated that this is not essential, and instead one or more of the processing systems 501, 503 can be distributed over geographically separate locations, for example by using processing systems provided as part of a cloud based environment.

In a preferred implementation, the server processing systems 501 may be provided as part of a cloud computing service and will communicate with other elements of the arrangement via the Internet 502. The use of user processing systems 503 in the form of client devices will allow users to monitor the composting process. Furthermore, users can interact with the server processing systems 501 or data stored on the database 611 to update data for use in the analysis of the monitoring data if necessary.

However, it will be appreciated that the above described arrangement is shown as an example only, and numerous other configurations may be used.

Further detailed implementation features of preferred embodiments of compost monitoring systems will now be described.

It will be appreciated that the compost monitoring system may be implemented using an “Internet of Things” methodology, by providing a compost monitoring device that may be wirelessly connected to a gateway device which in turn communicates with a cloud based server processing system to allow constant monitoring and processing using an analytical model, and user access to the monitoring data and information derived therefrom using a user processing system via a web portal or applications software.

The system is suitable for use in any small-scale composting operation to monitor and manage it using a compost monitoring device including gas based sensors for measuring properties of gas proximate to the compost, which wirelessly provides the monitoring data to the server processing systems, via the gateway device, for further analysis.

The system may be used to improve a composting process by constantly monitoring the decomposition, analysing this data to determine the condition of the compost, and using the monitoring data to drive improved decomposition and reduction of by-products of anaerobic decomposition such as methane, such as by recommending user interventions based on the determined conditions.

By continuously monitoring the composting process and other data sources, the analytical model can generate intervention instructions customised to unique compost operations. These instructions can optimise decomposition speed, minimise effort and eliminate any odours or pests, making composting efficient, compliant with regulations and producing a high-quality end product.

The key monitoring data obtained by the compost monitoring device includes temperature, humidity and levels of methane and/or other gas compounds, all using gas based sensors. However, in preferred implementations, the analytical model may also utilise other data including meteorological data and user entered data. The meteorological data may be obtained through open-source weather APIs and may include parameters including ambient temperature, ambient humidity, wind speed and rainfall. The user entered data may be in the form of a compost “profile”, which may require the user to answer a questionnaire to describe their compost set-up based on a number of options. In addition, the user may enter further data throughout the compost process, such as details of organic material added to the compost or specific interventions that may have been taken.

The analytical process performed by the server processing system may include generating models of the temperature, moisture and gas distribution throughout the pile. In some examples, the models may involve a three dimensional representation of the relevant parameters. Based on these models, custom instructions may be generated and provided to the user to prompt particular interventions for maintaining or restoring an optimal composting process. These instructions can be delivered on either a reactive or proactive basis.

A preferred embodiment of the compost monitoring device 300, as shown in FIGS. 3A to 3D, involves a modular configuration including a main sensor unit 310, a removable and rechargeable power supply unit 320, and a hollow stake 330 that penetrates into the surface 103 of the compost 101 and chimneys gas emitted from the compost 101 up to a sensor array 111 and a series of vents. The power supply unit may include a number of standard rechargeable batteries, such as AA NiMH batteries or any other suitable type, which can be conveniently charged using a charging functionality of the gateway device.

However, other examples of the compost monitoring device may be supported above the compost using a skirt that sits on the compost and funnels the gas towards the sensor array. In some examples, the compost monitoring device may be configured to be attached to a lid or side wall of the compost container (such as a bin or tumbler), and this attachment may be conveniently achieved using a magnetic coupling arrangement.

In any event, it will be appreciated that the above described compost monitoring device and compost monitoring system using the compost monitoring device may provide users with a convenient capability for monitoring a composting process and determining when to take interventions to optimise the composting results.

Throughout this specification and claims which follow, unless the context requires otherwise, the word “comprise”, and variations such as “comprises” or “comprising”, will be understood to imply the inclusion of a stated integer or group of integers or steps but not the exclusion of any other integer or group of integers. As used herein and unless otherwise stated, the term “approximately” means ±20%.

It must be noted that, as used in the specification and the appended claims, the singular forms “a,” “an,” and “the” include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

It will of course be realised that whilst the above has been given by way of an illustrative example of this invention, all such and other modifications and variations hereto, as would be apparent to persons skilled in the art, are deemed to fall within the broad scope and ambit of this invention as is herein set forth.

1) A compost monitoring device for use in monitoring properties of a body of compost, the compost monitoring device being configured to be positioned proximate to the body of compost in use, the compost monitoring device including:
a) a sensor array including sensors for sensing properties of gas proximate to the body of compost, the sensors including:
i) a temperature sensor for sensing a temperature of the gas;
ii) a humidity sensor for sensing a relative humidity of the gas; and
iii) a gas sensor for detecting a level of a gas compound emitted from the compost within the gas;
b) a wireless transceiver for wirelessly communicating with another device located remotely from the body of compost; and
c) a controller configured to:
i) obtain sensor data from the sensors;
ii) generate monitoring data using at least some of the sensor data; and
iii) cause the monitoring data to be transmitted to the other device using the wireless transceiver.

2) A compost monitoring device according to claim 1, wherein the gas sensor is a methane gas sensor for detecting a level of methane within the gas.

3) A compost monitoring device according to claim 1, wherein the gas sensor is configured for detecting respective levels of at least some of the following compounds within the gas:
a) methane;
b) hydrogen;
c) ethanol;
d) carbon monoxide; and
e) butane.

4) A compost monitoring device according to claim 1, wherein the gas sensor is a volatile organic compounds detector for detecting respective levels of a plurality of gas compounds emitted from the compost within the gas.

5) A compost monitoring device according to any one of claims 1 to 4, wherein the sensor array is positioned externally to a surface of the body of compost and exposed to the gas proximate to the body of compost in use.

6) A compost monitoring device according to claim 5, wherein the compost monitoring device includes a support arrangement for supporting the compost monitoring device relative to a surface of the body of compost in use.

7) A compost monitoring device according to claim 6, wherein the support arrangement is configured to engage with the surface of the body of compost in use.

8) A compost monitoring device according to claim 7, wherein the support arrangement is configured to at least one of:
a) rest on the surface of the body of compost in use; and
b) be partially inserted into the surface of the body of compost in use.

9) A compost monitoring device according to any one of claims 6 to 8, wherein the support arrangement is configured to define a hollow volume extending between the sensor array and the body of compost in use.

10) A compost monitoring device according to claim 9, wherein the support arrangement is configured so that gas compounds emitted from the surface of the body of compost are captured in the hollow volume and directed to the sensor array in use.

11) A compost monitoring device according to claim 9 or claim 10, wherein the support arrangement includes a hollow stake configured to be partially inserted into the surface of the body of compost.

12) A compost monitoring device according to claim 11, wherein the hollow stake includes:
a) a proximal end that surrounds the sensor array;
b) a distal end opposing the proximal end, the distal end being for insertion into the surface of the body of compost; and
c) a sidewall extending between the proximal end and the distal end and defining the hollow volume.

13) A compost monitoring device according to claim 12, wherein the sidewall is tapered from the proximal end to the distal end.

14) A compost monitoring device according to claim 12 or claim 13, wherein the sidewall of the hollow stake is vented.

15) A compost monitoring device according to claim 9 or claim 10, wherein the support arrangement includes a skirt extending from the sensor array and including a rim for engaging with the surface of the body of compost in use.

16) A compost monitoring device according to claim 15, wherein the skirt is flared such that the hollow volume expands outwardly from the sensor array to the rim.

17) A compost monitoring device according to claim 6, wherein the body of compost is provided in a container, and the support arrangement is configured to engage with a wall of the container to thereby support the compost monitoring device relative to the body of compost.

18) A compost monitoring device according to claim 17, wherein the support arrangement includes a magnetic coupling arrangement for magnetically coupling the compost monitoring device to the wall.

19) A compost monitoring device according to any one of claims 6 to 18, wherein the support arrangement is removably attached to the compost monitoring device.

20) A compost monitoring device according to any one of claims 5 to 19, wherein the compost monitoring device includes a removable filter proximate to the sensor array.

21) A compost monitoring device according to any one of claims 1 to 20, wherein the compost monitoring device includes a removable power supply unit including a rechargeable battery and a power supply interface for supplying power to the compost monitoring device.

22) A compost monitoring device according to claim 21, wherein the power supply interface includes at least one of:
a) electrical contacts; and
b) an inductive coupling interface.

23) A compost monitoring device according to any one of claims 1 to 20, wherein the compost monitoring device is configured to be powered by a separate solar power supply.

24) A compost monitoring device according to any one of claims 1 to 23, wherein the wireless transceiver is configured for wirelessly communicating with the other device using a Bluetooth wireless communication protocol.

25) A compost monitoring system for monitoring properties of a body of compost, the system including:
a) a compost monitoring device according to any one of claims 1 to 24, the compost monitoring device being positioned proximate to the body of compost in use; and
b) a gateway device located remotely from the body of compost but within wireless communications range of the compost monitoring device in use, the gateway device including:
i) a gateway wireless transceiver for wirelessly communicating with the compost monitoring device;
ii) a gateway network interface for communicating with a processing system via a communications network; and
iii) a gateway controller configured to:
(1) receive monitoring data from the compost monitoring device; and
(2) transmit at least some of the monitoring data to the processing system via the communications network.

26) A system according to claim 25, wherein the system further includes a server processing system, the server processing system being configured to:
a) receive monitoring data from the gateway device; and
b) determine a condition of the body of the compost based on received monitoring data.

27) A system according to claim 26, wherein the server processing system is configured to allow a user processing system to access monitoring data from the server processing system via the Internet.

28) A system according to claim 27, wherein the server processing system is configured to allow the user processing system to access the monitoring data using one of a web portal and an application programming interface.

29) A system according to any one of claims 26 to 28, wherein the server processing system is configured to generate one or more condition indicators indicative of the condition of the body of the compost and allow the user processing system to access the condition indicators.

30) A system according to any one of claims 26 to 29, wherein the server processing system is configured to:
a) determine whether any user interventions are required based on the condition of the body of the compost;
b) in the event of a determination that one or more user interventions are required, generate a user intervention indication indicative of the one or more user interventions; and
c) transmit the user intervention indication to the user processing system.

31) A system according to claim 30, wherein the user interventions include at least one of:
a) turning the body of compost;
b) adding moisture to the body of compost; and
c) adding organic matter to the body of compost.

32) A system according to any one of claims 26 to 31, wherein the server processing system is configured to obtain, from a meteorological data source, meteorological data for a geographical region corresponding to a location of the body of compost.

33) A system according to claim 32, wherein the server processing system is configured to obtain the meteorological data based on location data for at least one of the compost monitoring device, the gateway device, and the remote user device.

34) A system according to claim 32 or 33, wherein the meteorological data includes at least one of:
a) a relative humidity observation; and
b) a temperature observation.

35) A system according to any one of claims 32 to 34, wherein the server processing system is configured to determine the condition of the body of the compost based on received monitoring data and the meteorological data.

36) A system according to any one of claims 26 to 35, wherein the server processing system includes a plurality of processing devices in a cloud architecture.

37) A system according to any one of claims 25 to 36 wherein the gateway network interface includes a wireless network interface for wirelessly communicating with the server processing system using a wireless communications network.

38) A system according to any one of claims 25 to 37, wherein the gateway network interface includes a wireless modem for wirelessly communicating with the server processing system via the Internet.

39) A system according to any one of claims 25 to 37, wherein the gateway network interface includes a wireless access point for allowing a user device to wirelessly connect to the gateway device to allow the local user device to access monitoring data directly from the gateway device.

40) A system according to any one of claims 25 to 39, wherein the compost monitoring device includes a removable power supply unit including a rechargeable battery and the gateway device further includes a battery charging interface for recharging the battery of the removable power supply unit.
